(19) United States
(12) Patent Application Publication
(10) Pub. No.: US 2022/0257561 A1
(43) Pub. Date: Aug. 18, 2022

(54) COMPOSITIONS AND METHODS FOR IMPROVING WELLNESS

(71) Applicant: ADVANCED DELIVERY LABS LLC, Detroit, MI (US)

(72) Inventor: Jason P. TEJANI, Birmingham, MI (US)

(21) Appl. No.: 17/617,134

(22) PCT Filed: Jun. 8, 2020
(86) PCT No.: PCT/US20/36714

§ 371 Date: Dec. 7, 2021

Related U.S. Application Data
(60) Provisional application No. 62/858,965, filed on Jun. 7, 2019.
(60) Provisional application No. 62/928,356, filed on Oct. 30, 2019.

(51) Int. Cl. A61K 31/352; A61K 31/12; A61K 33/06; A61K 31/198; A61K 31/4415; A61K 31/714; A61K 9/00; A61P 25/20; A61K 47/46

(52) U.S. Cl. CPC A61K 31/352; A61K 31/12; A61K 33/06; A61K 31/198; A61K 47/46; A61K 31/714; A61K 9/0053; A61P 25/20; A61K 9/0095; A61K 31/4415

(57) ABSTRACT

A composition for improving wellness of a human is provided herein. The composition includes, but is not limited to, a flavonoid. The composition further includes, but is not limited to, a prodrug of cysteine. The composition further includes, but is not limited to, a pharmaceutically acceptable carrier. The pharmaceutically acceptable carrier includes, but is not limited to, an ionic liquid to form a solubilizing matrix. The flavonoid exhibits an improved solubility in the pharmaceutically acceptable carrier in the presence of the solubilizing matrix as compared to solubility of a flavonoid in a pharmaceutically acceptable carrier substantially free of the solubilizing matrix.

### CROSS-REFERENCE TO RELATED APPLICATIONS

This application claims priority to and all of advantages of U.S. Prov. Appl. No. 62/858,965, filed on 7 Jun. 2019, and U.S. Prov. Appl. No. 62/928,356, filed on 30 Oct. 2019, the contents of which are hereby incorporated by reference.

### TECHNICAL FIELD

The present disclosure relates to the field of compositions and methods for improving wellness of mammals, including humans. In particular, the present disclosure relates to compositions comprising one or more solubilized components, methods of forming the same, and methods of administering the same.

### BACKGROUND

Humans and animals are increasingly exposed to less than ideal conditions including, but not limited to, poor nutrition, unsatisfactory rest, and excessive stress. These conditions result in a variety of metabolic and hormonal imbalances which in turn reduce wellness and overall well-being. In particular, humans may exhibit headache, nausea, disruption of mental awareness, muscle strain and soreness, aches, eye strain and difficulty focusing, and fatigue.

One example of a less than ideal condition includes shift work sleep disorder (SWSD). SWSD is a circadian rhythm sleep disorder affecting people whose work hours overlap with the typical sleep period. Symptoms include insomnia, excessive fatigue, headache, malaise, nausea, and a generalized altered sense of well-being. Examples of common professions that may experience SWSD include, but are not limited to, healthcare professionals, police officers, firefighters, military personnel, rideshare operators, factory workers, travelers, and dependent care givers (e.g., parents of an infant).

Another example of a less than ideal condition includes consumption of alcohol which can result in a “hangover”. Hangover is a complex phenomenon resulting from not only the ethyl alcohol (ethanol) component of alcoholic beverages but also from the milieu of organic and non-organic compounds found in particular alcoholic beverages, such as beer, wine, and liquors, and their metabolites. Hangover is best described as a syndrome with a wide spectrum of symptoms in response to highly variable amounts of alcohol consumption and individual (idiosyncratic) reactions. Further, the consumption of alcohol can negatively impact a human's ability to obtain a restorative sleep. Typical symptoms of hangover include headache, malaise, gastrointestinal upset, nausea, and a generalized altered sense of well-being. Historically, many commonly available analgesic drugs as well as folklore remedies have been used in the treatment of hangovers resulting from the consumption of alcoholic beverages. However, no known approach has proven to be adequate.

Another example of a less than ideal condition includes consumption of a food which can result in an adverse event due to a sensitivity to the food. A food sensitivity, or a food intolerance occurs when a human has difficulty digesting a particular food. This can lead to symptoms such as intestinal gas, abdominal pain or diarrhea. Examples of common food intolerances include, but are not limited to, dairy, gluten, caffeine, salicylates, amines, fermentable oligo-, di-, mono-saccharides and polyols (FODMAPs), sulfites, and fructose.

Attempts have been made to treat illnesses and disorders, such as the aforementioned disorders, with naturally occurring products. Such products were initially in their natural state, such as leaves, berries, roots, tree cuttings, and extracts. With the advance of science, and greater understanding of chemistry, various active substances of naturally occurring products have been isolated for use by humans.

However, formulators have failed to take a holistic approach when formulating compositions including active substances. In particular, many of the aforementioned disorders result from a combination of unrelated metabolic and/or hormonal pathways. Furthermore, most orally administered active substances fail to pass from the digestive system into a subject's bloodstream and thus are not effective. Even if the substance makes it into a subject's bloodstream, the concentration of these substances in blood typically varies over time. There may be a large increase in concentration shortly after orally administering the substance, followed by a steady decrease in concentration as the substance is metabolized.

Accordingly, it is desirable to provide improved compositions and methods for improving wellness. Furthermore, other desirable features and characteristics will become apparent from the subsequent summary and detailed description and the appended claims, taken in conjunction with the foregoing technical field and background.

### BRIEF SUMMARY

In certain embodiments, a composition for improving wellness of a human is provided herein. The composition includes, but is not limited to, a flavonoid. The composition further includes, but is not limited to, a prodrug of cysteine. The composition further includes, but is not limited to, a pharmaceutically acceptable carrier. The pharmaceutically acceptable carrier includes, but is not limited to, an ionic liquid to form a solubilizing matrix. The flavonoid exhibits an improved solubility in the pharmaceutically acceptable carrier in the presence of the solubilizing matrix as compared to solubility of a flavonoid in a pharmaceutically acceptable carrier substantially free of the solubilizing matrix.

Additional features, advantages, and embodiments of the invention may be set forth or apparent from consideration of the following detailed description, drawings, and claims. Moreover, it is to be understood that both the foregoing summary of the invention and the following detailed description are exemplary and intended to provide further explanation without limiting the scope of the invention as claimed.

### BRIEF DESCRIPTION OF THE DRAWINGS

The present invention will hereinafter be described in conjunction with the following drawing figures, wherein like numerals denote like elements, and

FIG. 1 is a graphical view illustrating experimental results for non-limiting embodiment of a composition administered to subjects;

FIG. 2 is another graphical view illustrating experimental results for non-limiting embodiment of a composition administered to subjects; and

FIG. 3 is another graphical view illustrating experimental results for non-limiting embodiment of a composition administered to subjects.

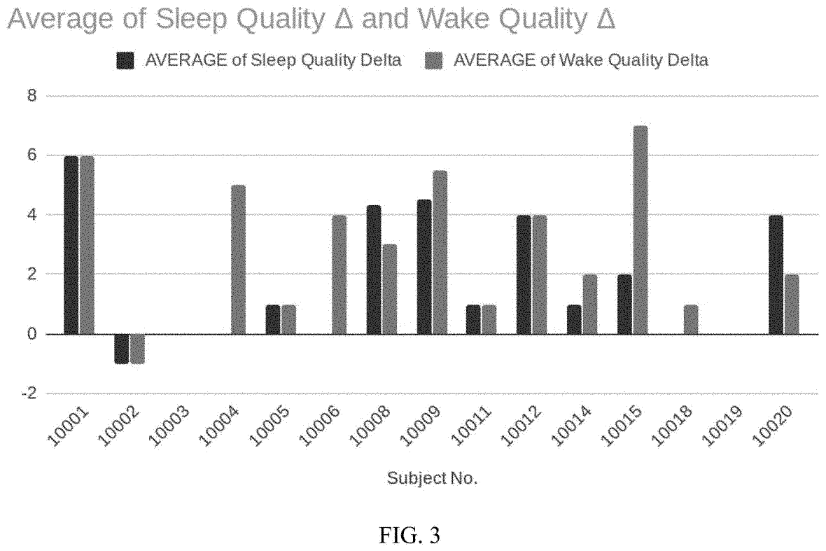

### DETAILED DESCRIPTION

Except in the examples, or where otherwise expressly indicated, all numerical quantities in this description indicating amounts of material or conditions of reaction and/or use are to be understood as modified by the word “about” in describing the broadest scope of the invention. Practice within the numerical limits stated is generally preferred. Also, unless expressly stated to the contrary: percent, “parts of,” and ratio values are by weight; the description of a group or class of materials as suitable or preferred for a given purpose in connection with the invention implies that mixtures of any two or more of the members of the group or class are equally suitable or preferred; description of constituents in chemical terms refers to the constituents at the time of addition to any combination specified in the description, and does not necessarily preclude chemical interactions among the constituents of a mixture once mixed; the first definition of an acronym or other abbreviation applies to all subsequent uses herein of the same abbreviation and applies mutatis mutandis to normal grammatical variations of the initially defined abbreviation; and, unless expressly stated to the contrary, measurement of a property is determined by the same technique as previously or later referenced for the same property.

It must also be noted that, as used in the specification and the appended claims, the singular form “a,” “an,” and “the” comprise plural referents unless the context clearly indicates otherwise. For example, reference to a component in the singular is intended to comprise a plurality of components.

Throughout this application, where publications are referenced, the disclosures of these publications in their entireties are hereby incorporated by reference into this application to more fully describe the state of the art to which this invention pertains.

The following detailed description is merely exemplary in nature and is not intended to limit the invention or the application and uses of the invention. Furthermore, there is no intention to be bound by any theory presented in the preceding background or the following detailed description.

A composition for improving wellness of a human, kits for the same, and methods for and relating to the same, are provided herein. The composition may include a flavonoid and an ionic liquid. In various embodiments, the composition includes a therapeutically effective amount of the compound(s) described herein, or a pharmaceutically acceptable salt thereof, pharmaceutically active metabolite thereof, pharmaceutically acceptable prodrug thereof, or pharmaceutically acceptable solvate thereof. In certain embodiments, compositions provided herein further include a pharmaceutically acceptable diluent, excipient and/or binder.

In other embodiments, the composition may include a flavonoid, a magnesium-containing compound, and a prodrug of cysteine. In these and other embodiments, the composition may include the ionic liquid, which includes the magnesium-containing compound.

The composition may further include a pharmaceutically acceptable carrier. The pharmaceutically acceptable carrier may include an emulsifier dispersed throughout the pharmaceutically acceptable carrier to form a solubilizing matrix. Alternatively, the pharmaceutically acceptable carrier may include the ionic liquid to form a solubilizing matrix. The flavonoid exhibits an improved solubility in the pharmaceutically acceptable carrier in the presence of the solubilizing matrix as compared to solubility of a flavonoid in a pharmaceutically acceptable carrier substantially free of the solubilizing matrix.

In certain embodiments, the composition is useful for treating a sleep-related condition or disorder. Examples of sleep-related conditions or disorders may include, but are not limited to, sleep apnea, insomnia, sleep deprivation (e.g., due to dependent care or infant care), circadian rhythm sleep-wake disorder (e.g., due to shift work or jet lag), parasomnias, and sleep movement disorders. In various embodiments, the composition is administered no greater than 120 minutes, no greater than 90 minutes, no greater than 60 minutes, no greater than 45 minutes, or no greater than 30 minutes, before attempting to sleep.

In certain embodiments, the composition is substantially free of sedatives. Non-limiting examples of sedatives include melatonin, valerian root, 5-HTP, benzodiazepines, barbiturates, hypnotics, and opioids. In various embodiments, the composition is substantially free of stimulants. Non-limiting examples of stimulants include caffeine, nicotine, and amphetamines.

Gamma amino butyric acid (GABA) is believed to be one of the more prevalent neurotransmitters in the brain and is usually responsible for inhibition. GABA release is often seen as a way to shut down or down regulate neurons. One of the main areas that GABA may be involved in the sleep-wake cycle is at the posterior hypothalamus. The stimulation of the neurons in the posterior hypothalamus are known to contribute to wakefulness. Without being bound by theory, it is believed that increased levels of GABA in the posterior hypothalamus contribute to inducing sleep.

Glutamate is believed to be the most common neurotransmitter in the brain and one of the main excitatory neurotransmitter. Glutamate is also a precursor for GABA which is the main inhibitory neurotransmitter in the brain. Glutamate is involved in a variety of areas of the sleep-wake cycle. Glutamatergic input to the oral part of the pontine reticular formation is thought to regulate sleep duration. It has also believed that glutamatergic inputs in the posterior hypothalamic region help regulate both REM sleep and wakefulness. Without being bound by theory, it is believed that the composition as contemplated herein favors increased GABA(A) receptor binding while reducing glutamate activity.

As well known in the art, circadian rhythm and homeostatic sleep drive work together to regulate sleep-wake cycles. Circadian rhythms direct a wide variety of functions from daily fluctuations in wakefulness to body temperature, metabolism, and the release of hormones. Circadian rhythms control the subject's timing of sleep and cause the desire to rest at night and wake in the morning without an alarm. The body's biological clock, which is based on a roughly 24-hour day, controls most circadian rhythms. It is believed that the balance between cortisol release and melatonin secretion can influence the circadian rhythm of a subject. In particular, melatonin secretion may occur during low cortisol release.

In contrast, sleep-wake homeostasis is independent of the actual time of day and keeps track of the need for sleep. The homeostatic sleep drive reminds the body to sleep after a certain amount of time has passed being awake and regulates sleep intensity. This sleep drive increases every hour that the subject is awake and causes the subject to sleep longer and more deeply after a period of sleep deprivation. Two of the key molecules in regulation of the homeostatic sleep drive is the inhibitory neurotransmitter GABA and the excitatory neurotransmitter glutamate. It is believed that glutamate and GABA regulate the excitation/inhibition balance in the brain.

When under normal sleep conditions (i.e., normal sleep-wake cycle), cortisol should be naturally low at night, allowing melatonin to become dominant and GABA as an inhibitory neurotransmitter becomes dominant over glutamate. In particular, during wake, it is believed that cortisol is dominant over melatonin and glutamate is dominant over GABA. During sleep, it is believed that melatonin is dominant over cortisol and GABA is dominant over glutamate.

In contrast, subjects that suffer from disruptions in sleep-wake cycles experience both the homeostatic sleep drive (the accumulating need for sleep) and the circadian drive (the normal sleep-wake cycle) to sleep. Sleep-wake cycles can be disrupted in a number of ways, including working at night and traveling between time zones. In particular, during wake, it is believed that melatonin increases relative to cortisol and GABA increases relative to glutamate. During sleep, it is believed that cortisol remains elevated relative to melatonin and glutamate remains elevated relative to GABA.

In other embodiments, the composition is useful for treating food-related conditions. Examples of food-related conditions may include, but are not limited to, over consumption of alcohol, intolerances to foods (e.g., Crohn's disease, inflammatory bowel disease, irritable bowel syndrome, and intolerances to dairy, gluten, caffeine, salicylates, amines, fermentable oligo-, di-, mono-saccharides and polyols (FODMAPs), sulfites and fructose).

Acetaldehyde (CH₃CHO, also referred to as ethanol) is an aldehyde that is found in nature and also produced by the partial oxidation of ethanol in the liver by an enzyme alcohol dehydrogenase (ADH). Acetaldehyde is considered to be a contributing constituent that causes hangovers from alcohol consumption. Acetaldehyde dehydrogenase (ALDH) metabolizes acetaldehyde to acetate.

The liver acts to clear alcohol and acetaldehyde from the body. Alcohol consumption (and particularly over consumption) leads to liver stress and L-Glutathione depletion, leaving the individual with a temporarily overtaxed liver that cannot properly clear the body of toxins for a period of time during and after drinking. This over-taxation of the liver can lead to a buildup of lactic acid which is known to be related to feelings of lethargy, e.g., and is thereby also considered to be another physiological factor contributing to hangovers.

In particular, the first step is the breakdown of ethanol to acetaldehyde, a toxic compound that can promote cell and tissue damage. From there, acetaldehyde is metabolized by the enzyme acetaldehyde dehydrogenase, leading to the production of glutathione, an antioxidant. However, when ethanol intake is high, acetaldehyde dehydrogenase activity is insufficient to process the amount of acetaldehyde that accumulates, which can lead to acetaldehyde buildup in the liver. The subsequent acetaldehyde accumulation is believed to mediate acute hangover symptoms, such as headache and nausea. As levels of acetaldehyde rise, levels of glutathione decrease. This is partially because a lower amount of acetaldehyde is being broken down by the acetaldehyde dehydrogenase enzyme. Additionally, glutathione may be depleted in the presence of excessive acetaldehyde because it forms conjugates with acetaldehyde. The increase in these glutathione-acetaldehyde conjugates has the potential to reduce glutathione antioxidant activity, which could further contribute to oxidative stress that produces more severe hangover symptoms. Without being bound by theory, it believed that the composition as contemplated herein improves the activity of alcohol dehydrogenase and acetaldehyde dehydrogenase, and reduces binding of alcohol to GABA(A) receptors.

The individual components/compounds of the composition will now be described in detail.

### Flavonoids

Flavonoids include a group of natural substances (commonly referred to as nutraceuticals) with phenolic structures that may be found, for example, in fruits, vegetables, grains, bark, roots, stems, flowers, tea and wine. In certain embodiments, flavonoids exhibit anti-oxidative, anti-inflammatory, anti-mutagenic, and anti-carcinogenic properties coupled with their capacity to modulate key cellular enzyme function. Flavonoids may inhibit various enzymes including, but not limited to, xanthine oxidase (XO), cyclo-oxygenase (COX), lipoxygenase, and phosphoinositide 3-kinase.

Flavonoids are typically classified according to their chemical structure, and may be subdivided into 6 subgroups: flavonols, such as, quercetin, kaempferol, myricetin, isorhamnetin; flavones, such as, luteolin, apigenin, tangeritin, polymethoxylated flavones, tageretin, nobiletin, and sinensetin; flavanones, such as, hesperitin, naringenin, and eriodictyol; flavan-3-ols, such as, catechin, gallocatechin, epicatechin, epigallocatechin, epicatechin 3-gallate, epigallocatechin 3-gallate, theaflavin, theaflavin 3-gallate, theaflavin 3′-gallate, theaflavin 3,3′ digallate, and thearubigins; isoflavones, such as, genistein, daidzein, and glycitein; and anthocyanidins, such as, cyanidin, delphinidin, malvidin, pelargonidin, peonidin, and petunidin. Examples of flavonoids may include, but are not limited to, flavones, flavonols, flavanones, flavanonols, flavanols, catechins, anthocyanins, chalcones, and combinations thereof.

Flavonols are flavonoids with a ketone group and may be building blocks of proanthocyanins. Flavonols may occur abundantly in a variety of fruits and vegetables, including onions, kale, lettuce, tomatoes, apples, grapes and berries. Examples of suitable flavonols include, but are not limited to, quercetin, kaempferol, myricetin, isorhamnetin, and fisetin.

Flavanonols, also called dihydroflavonols, flavan-3-ols, or catechins, are 3-hydroxy derivatives of flavanones, in particular, including a 3-hydroxy-2,3-dihydro-2-phenylchromen-4-one backbone. Flavanonols are a highly diversified and multisubstituted subgroup. Examples of suitable flavanonols include, but are not limited to, ampelopsin (also known as dihydromyricetin), taxifolin (also known as dihydroquercetin), aromadedrin (also known as dihydrokaempferol), and engeletin (also known as dihydrokaempferol-3-rhamnoside). Various examples of flavonoids may be found in Panche, A. N., Diwan, A. D., & Chandra, S. R. (2016). Flavonoids: an overview. Journal of nutritional science, 5, e47. doi:10.1017/jns.2016.41, which is incorporated by reference as if fully set forth herein.

Other examples of suitable flavonoids includes, but are not limited to, 6-OH-Luteolin 4′-methyl ether-7-(2″-a-rhamnoside-6′″-acetyl-b-glucoside), 6-OHLuteolin 7-(6″-(E)-caffeoyl)-b-glucoside, Isoscutellarein 7-(2″-(6′″-acetyl)-b-allosyl)-bglucoside, Isoscutellarein 4′-methyl ether-7-(2″-(6′″-acetyl)-b-allosyl)-b-glucoside, Apigenin 4′-(2″-(2′″-feruloyl-glucuronyl)-glucuronide), Apigenin 7-glucuronide-4′-(2″-(2′″-feruloyl-glucuronyl)-glucuronide), Apigenin 7-glucuronyl-4′-(2-(2′″-E-pcoumaroyl-glucuronyl)-glucuronide), Luteolin 3′-b-glucoside-4′-(2″-a-rhamnosyl-bglucoside), Luteolin 3′,4′-di-b-glucoside, 5,7,4′-tri-OH-3′-OMe-Flavone 8-C-(2″-Ob-glucosyl-b-xyloside), 5,7-di-OH-3′-OMe-4′-Acetoxyflavone 8-C-(2″-O-b-glucosylb-xyloside), Iso-orientin 3′-methyl ether, 8-C-p-OH-Benzoyl-isovitexin 4′-glucoside, Apigenin 8-C-(2″-(4′″-acetyl-rhamnosyl)-glucoside), Spinosin, 6′″-Feruloyl-spinosin, Isoscoparin 7-glucoside, Carlinoside, Kaempferol 3-(6″-aarabinosyl-glucoside), Kaempferol 3-(6″-a-arabinosyl-glucoside)-7-glucoside, Kaempferol 3-(2″-rhamnosyl-6″-malonyl-glucoside), Kaempferol 3-glucoside-7-(2″-(6′″-p-coumaroyl-glucosyl)-glucoside), 8-OMe-Kaempferol 3-(6″-malonyl-glucoside), Quercetin, Quercetin chalcone, Quercetin 4′-glucoside, Quercetin 3′-xyloside, Myricetin 3-(2″-acetyl-rhamnoside), Quercetin 3,4′-diglucoside, Isorhamnetin, Isorhamnetin 3-rutinoside, Quercetin 3,7,4′-triglucoside, Isorhamnetin 3,7-diglucoside, Myricetin 3-(2″-rhamnosyl-glucoside), Myricetin 3′-(6″-p-coumaroyl-glucoside), Myricetin 7-(6″-galloyl-glucoside), Laricitrin 3-a-arabinofuranoside, Laricitrin 3-glucoside, Syringetin 3-(5″-glucosyl-a-arabinofuranoside), Syringetin 3-(6″-acetyl-glucoside), Syringetin 3-robinobioside, Syringetin 6-C-glucoside, 6,3′-di-OH-4,4′-di-OMe-5-Me-Aurone, 4,6,3′,4′-tetra-OMe-Aurone (Z-form), 4,6,3′,4′-tetra-OMe-Aurone (E-form), 6,3′,4′-tri-OH-4-OMe-5-Me-Aurone, Maesopsin, Maesopsin 6-O-glucoside (two diastereoisomers), Licoagroaurone, 3′-formyl-4′,6′-di-OH-2′-OMe-5-Me-Chalcone, Chalcononaringenin 2′,4′-diglucoside, 2′,4′-diOH-4′-OMe-6′-glucoside Dihydrochalcone, 2′-OH-3′,4′,6′-tri-OMe-Dihydrochalcone, Pelargonidin 3-glucoside-5-(6′″-acetyl-glucoside), Pelargonidin 3-(6″-feruloylglucoside)-5-(6′″-malonyl-glucoside), Cyanidin 3-(6″-malonyl-glucoside), Cyanidin 3-rutinoside, Cyanidin 3-(2″,3″-digalloyl-glucoside), Cyanidin 3,4′-diglucoside, Delphinidin 3-(6″-acetyl-galactoside), Delphinidin 3′-(2″-galloyl-6″-acetyl-galactoside), Peonidin 3-rutinoside, Petunidin 3,7-diglucoside, Petunidin 3-(6″-E-p-coumaroyl-glucoside)-5-(6′″-malonyl-glucoside), Malvidin 3-(6″-E-p-coumaroyl-glucoside)-5-glucoside, Malvidin 3-(6″-Z-p-coumaroyl-glucoside)-5-glucoside, Malvidin 3-rutinoside-5-glucoside, Malvidin 3-(6″-(4″ ″-malonylrhamnosyl)-glucoside)-5-(6′″-malonyl-glucoside), Apigeninidin 5-glucoside, Luteolinidin 5-glucoside, Carboxypyrano Pelargonidin 3-glucoside, Carboxypyrano Cyanidin 3-glucoside, Carboxypyrano Cyanidin 3-(6″-malonylglucoside) Carboxypyrano Malvidin 3-glucoside, Judaicin 7-(6″-acetylglucoside), Tectorigenin 4′-(6″-glucosyl-glucoside), 7-OH-6′-OMe-3′,4′-methylenedioxyisoflavone 7-glucoside, Irisjaponin A, Irisjaponin B, Junipegenin B, Matteucinol 7-(6″-apiofuranosyl-b-glucoside), Hesperitin 7-(2″-galactosyl-6″-rhamnosyl-glucoside), Persicogenin 5,3′-di-OH-7,4′-di-OMeflavanone, Naringenin 7-glucoside, Naringenin 7-(6″-galloyl-glucoside), Taxifolin 4′-glucoside, Aromadendrin 7-glucoside, Ampelopsin 7-glucoside, 2″-Accallunin, 2R,3R-trans-aromadendrin 7-(6-(4′″-OH-2′″-methylenebutanoyl)-glucoside), (2R,3 S)-(b)-3′,5-di-OH-4′, 7-di-OMe-Dihydroflavonol, 3-Desoxycallunin, Catechin 3-(6″-cinnamoyl-glucoside), Catechin 3-(2″-cinnamoyl-glucoside), Catechin 3-(2″,6″-dicinnamoyl-glucoside), Anadanthoside, Cajanin, Indicanine C, 6-(1,1-di-Me-allyl)-7,4′-di-OH-Flavan, 3-(4′-hydroxyphenyl)-5-methoxy-6-(3,3-dimethylallyl)-2″,2″-dimethylchromene-(5″,6″:8,7)-3-(propyl-2-one)-4H-1-benzo-2,3-Dihydropyran-2,4-dione, Maackianin 3-(6″-malonyl-glucoside), 3,4:8,9-Dimethylenedioxy-pterocarpan, Usararotenoid C, 12a-Epimillettosin, (b)-Usararotenoid-B, Rhuschalcone 1, Puerarin (Daidzein 8-C-glucoside), Calycosin, Isoneorautenol, Erybraedin A, and combinations thereof.

In certain embodiments, the flavonoid includes a quercetin-containing compound, a GABA(A) receptor antagonist, or a combination thereof. The quercetin-containing compound, a GABA(A) receptor antagonist, or a combination thereof, may be included in the composition in a therapeutically effective amount, or as a pharmaceutically acceptable salt thereof, pharmaceutically active metabolite thereof, pharmaceutically acceptable prodrug thereof, or pharmaceutically acceptable solvate thereof.

### Quercetin-Containing Compound

Quercetin is a flavonoid that forms the “backbone” for many other flavonoids, including the citrus flavonoids rutin, hesperidin, naringin and tangeritin. Quercetin exhibits anti-inflammatory activity because of direct inhibition of several initial processes of inflammation. For example, quercetin inhibits both the manufacture and release of histamine and other allergic/inflammatory mediators. In addition, quercetin exerts potent antioxidant activity and vitamin C-sparing action.

In various embodiments, quercetin suppresses cortisol release. Quercetin may also exhibit an effect on the sleep-wake cycle of humans and animals. In particular, quercetin may decrease REM sleep, while increasing non-REM sleep. Without being bound to theory, it is believed that the GABA(A) receptor may be impacted by quercetin thereby leading to a decrease REM sleep. As described below, in certain embodiments, the composition as contemplated herein prevents the decrease of REM sleep resulting from the quercetin.

Quercetin, as a phenolic compound, may reduce acetaldehyde accumulation by improving alcohol metabolism thereby reducing the effects of a “hangover” from alcohol consumption. In particular, as acetaldehyde accumulated in the body, a reduction of antioxidant activity within the body occurs. Without being bound by theory, it is believed that quercetin promotes an increase in acetaldehyde dehydrogenase activities thereby reducing the accumulation of acetaldehyde.

In an exemplary embodiment, the quercetin-containing compound is quercetin. The quercetin-containing compound may not be soluble in water and therefore may exhibit poor bioavailability in humans and animals. As introduced above, the quercetin-containing compound exhibits improved solubility in the solubilizing matrix which may result in improved bioavailability in humans and animals. Alternatively, or in addition to the solubilizing matrix, the composition may include a water-soluble complex of the quercetin-containing compound for improving solubility in the pharmaceutically acceptable carrier which may result in improved bioavailability in humans and animals.

The composition may include the quercetin-containing compound in an amount of from about 1 to about 10,000 mg, alternatively from about 10 to about 5,000 mg, or alternatively from about 100 to about 1,000 mg, based on a total weight of 100,000 mg. In certain embodiments, the quercetin-containing compound is quercetin. Quercetin is commercially available.

In other embodiments, a metabolite of the quercetin-containing compound, such as isorhamnetin, may be included in the composition. The composition may include the metabolite of the quercetin-containing compound in an amount of from about 1 to about 10,000 mg, alternatively from about 10 to about 5,000 mg, or alternatively from about 100 to about 1,000 mg, based on a total weight of 100,000 mg. Isorhamnetin is commercially available.

### GABA(A) Receptor Antagonist

The GABA(A) receptor antagonist may be a mild antagonist of the GABA(A) receptor. In various embodiments, the GABA(A) receptor antagonist prevents binding of various compounds to the GABA(A) receptor while still permitting binding of GABA. The GABA(A) receptor antagonist may include ampelopsin (also commonly referred to as dihydromyriceten). Ampelopsin is a flavonoid compound found in plants such the Ampelopsis species japonica, megalophylla and grossedentata, Cercidiphyllum japonicum, Hovenia dulcis, Rhododendron cinnabarinum, some Pinus species, and some Cedrus species, as well as in Salix sachalinensis. Ampelopsin may exhibit a number of biological and pharmacological properties including antimicrobial, anti-inflammatory, antioxidative, anti-hypertensive, hepatoprotective and anticarcinogenic activities in addition to providing cough relief.

Ampelopsin may promote restorative sleep by binding to receptors in the nervous system. In particular, the release of gamma-aminobutyric acid (GABA) and subsequent binding to GABA(A) receptors has been associated with an increase in slow-wave sleep relative to wake and rapid eye movement sleep. Without being bound by theory, it is believed that the GABA(A) receptor antagonist, such as ampelopsin, may also bind to the GABA(A) receptors thereby increasing slow-wave sleep relative to wake and rapid eye movement sleep. Further, it is also believed that the binding of GABA(A) receptors with the GABA(A) receptor antagonist, such as ampelopsin, prevents the decrease of REM sleep by the quercetin-containing compound, such as quercetin, while still permitting an increase in non-REM sleep by quercetin.

Ampelopsin may improve metabolic activity of alcohol in the liver and may prevent alcohol from reaching receptors in the brain, which reduces that feeling of intoxication and hangovers. In particular, ampelopsin is understood to increase the rate at which alcohol and acetaldehyde are removed by the liver, and to reduce at least some of the negative effects of alcohol on the brain. Without being bound by theory, it is believed that ampelopsin binds to the GABA(A) receptor which are the same receptors in the nervous system as alcohol binds to. Ampelopsin may be taken prior to, and after, the consumption of alcohol.

In other embodiments, the composition includes a GABA(A) receptor antagonist that may not be a flavonoid. Examples of suitable GABA(A) receptor antagonists include, but are not limited to, bicuculline, securinine, metrazol, flumazenil, thujone, Ginkgo biloba, kudzu, muira puama, Piper methysticum L.f, Zizyphus jujuba Mill var. spinosa, Valeriana officinalis L., Hypericum montbretii Spach, Pinus massoniana Lamb., Scutellaria baicalensis Georgi, Atractylodes macrocephala Koidz., Ipomoea tyrianthina (Pelletan) Ledeb. ex Steud, Ternstroemia lineata DC., Rhus parviflora Roxb., Dimocarpus longan Lour, Crassocephalum bauchiense (Hutch.) Milne-Redh, Chrysanthemum morifolium Ramat., Dorstenia arifolia Lam., Magnolia officinalis Rehder & E. H. Wilson, Glycyrrhiza glabra L., Ecklonia cava Kjellman, Melissa officinalis L., 4-amino-3-phenylbutyric acid, and combinations thereof. Other examples of the GABA(A) receptor antagonist that may not be a flavonoid may be found in Johnston G. A. (2013). Advantages of an antagonist: bicuculline and other GABA antagonists. British journal of pharmacology, 169(2), 328-336. doi:10.1111/bph.12127, which is incorporated by reference as if fully set forth herein.

Ampelopsin or the GABA(A) receptor antagonist may not be soluble in water and therefore may exhibit poor bioavailability in humans and animals. As introduced above, the ampelopsin or the GABA(A) receptor antagonist exhibits improved solubility in the solubilizing matrix which may result in improved bioavailability in humans and animals. Alternatively, or in addition to the solubilizing matrix, the composition may include a water-soluble complex of ampelopsin or the GABA(A) receptor antagonist for improving solubility in the pharmaceutically acceptable carrier which may result in improved bioavailability in humans and animals.

The composition may include ampelopsin in an amount of from about 1 to about 10,000 mg, alternatively from about 10 to about 5,000 mg, or alternatively from about 100 to about 1,000 mg, based on a total weight of 100,000 mg. Ampelopsin is commercially available.

In other embodiments, a metabolite of ampelopsin, such as 5,7,3′,5′-tetrahydroxyflavanonol, 5,7,3′,5′-tetrahydroxy-4′-methoxyflavanonol, 5,7,4′,5′-tetrahydroxy-3′-methoxyflavanonol, dihydromyricetin-O-5-β-D -glucuronide, (2R,3S)-5,7,3′,4′,5′-pentahydroxyflavanonol; 3,4,5,7,3′,4′,5′-hepthydroxyflavan, and 5,7,3′,4′,5′-pentahydroxyflavanone, may be included in the composition. The composition may include the metabolite of ampelopsin in an amount of from about 1 to about 10,000 mg, alternatively from about 10 to about 5,000 mg, or alternatively from about 100 to about 1,000 mg, based on a total weight of 100,000 mg.

### Prodrug of Cysteine

The prodrug of cysteine may be any compound known in the art that can be metabolized to form cysteine. Examples of suitable prodrugs of cysteine include, but are not limited to, N-acetyl cysteine, L-2-oxothiazolidine-4-carboxylic acid, and a combination thereof. In certain embodiments, the prodrug of cysteine is N-acetyl cysteine.

N-acetyl cysteine exhibits antioxidant activity. The cysteine moiety of N-acetyl cysteine combines with glutamate and glycine, all of which are precursors in the production of glutathione. In the production of glutathione, cysteine is the rate-limiting step. Glutathione, in turn, is a major endogenous antioxidant. In fact, it is the most generic cellular antioxidant in the body. Because of its antioxidant activity, glutathione is essential for the immune system to exert its full potential. Without being bound by theory it is believed that an increase in glutathione promotes restorative sleep.

N-acetyl cysteine may also promote liver support and reduce acetaldehyde toxicity that causes many hangover symptoms by increasing the body's production of the glutathione. In particular, N-acetyl cysteine is a natural precursor to L-glutathione, which is an enzyme in the liver that breaks down acetaldehyde. In some embodiments, for example, the N-acetyl cysteine (e.g., or any of its functional substitutes such as SAM-e or L-glutathione, or other) is provided by the composition to counteract the toxic effects of acetaldehyde. In various embodiments, N-acetyl cysteine is only administered prior to the consumption of alcohol.

The composition may include the prodrug of cysteine in an amount of from about 1 to about 10,000 mg, alternatively from about 10 to about 5,000 mg, or alternatively from about 100 to about 1,000 mg, based on a total weight of 100,000 mg. The prodrug of cysteine is commercially available.

### L-Theanine

In certain embodiments, the composition further includes L-theanine. L-theanine is an amino acid that may be derived from green tea. L-theanine may promote the generation of alpha brain waves, which are produced when the brain is in a relaxed state. L-theanine may enhance levels of the neurotransmitters serotonin, dopamine, and GABA. In addition, L-theanine may block glutamate receptors, preventing excessive nerve cell excitation that would otherwise occur. L-theanine may be beneficial in combating fatigue and anxiety symptoms related to stress. The composition may include the L-theanine in an amount of from about 1 to about 10,000 mg, alternatively from about 10 to about 5,000 mg, or alternatively from about 100 to about 1,000 mg, based on a total weight of 100,000 mg for the composition. L-theanine is commercially available.

### 5-Hydroxytryptophan (“5-HTP”)

In certain embodiments, the composition further includes 5-HTP. 5-HTP is a precursor to the neurotransmitter, serotonin. The composition may include the 5-HTP in an amount of from about 1 to about 10,000 mg, alternatively from about 10 to about 5,000 mg, or alternatively from about 100 to about 1,000 mg, based on a total weight of 100,000 mg for the composition. 5-HTP is commercially available.

### Gamma-Aminobutyric Acid (“GABA”)

In certain embodiments, the composition further includes gamma-aminobutyric acid (“GABA”). As described above, gamma-aminobutyric acid (GABA) is believed to be one of the more prevalent neurotransmitters in the brain and is usually responsible for inhibition. GABA release is often seen as a way to shut down or down regulate neurons. One of the main areas that GABA may be involved in for the sleep-wake cycle is the posterior hypothalamus. The stimulation of the neurons in the posterior hypothalamus are known to contribute to wakefulness. Without being bound by theory, it is believed that increased levels of GABA in the posterior hypothalamus contribute to inducing sleep. The composition may include the GABA in an amount of from about 1 to about 10,000 mg, alternatively from about 10 to about 5,000 mg, or alternatively from about 100 to about 1,000 mg, based on a total weight of 100,000 mg for the composition. The GABA is commercially available.

### Curcuminoid

In certain embodiments, the composition further includes a curcuminoid. The curcuminoid may be an extract of turmeric, such as a turmeric extract with 95% curcuminoid that is prepared from the root or rhizome of the Curcuma longa plant (common names: Curcuma, Turmeric, Ukon, Goeratji, Kakoenji, Koenjet, Kondin, Kunir, Kunyit, Oendre, Rame, Renet, Temu kuning, Temu kunyit, Tius. Curcumin). C. longa is a perennial plant native to India.

In various embodiments, the curcuminoid includes curcumin. Curcumin is a potent extract of the root and has been attributed a wide range of therapeutic benefits. Curcuminoids and/or turmeric extracts are useful as an antioxidant, anti-inflammatory, anti-mutagenic agent, anti-cancer agent, cholagogueue, depurative, diuretic, fumitory, hemostatic agent, hepatoprotective agent, lactagogue, stomachic, tonic, and vulnerary.

Curcumin exhibits antioxidant activity. In some embodiments, curcumin inhibits cortisol secretion stimulated by stress responses. As described above, melatonin secretion occurs during low cortisol release. Therefore, it is believed that curcumin may promote melatonin release by inhibiting cortisol secretion.

Turmeric preparations including curcuminoids may also be useful to protect the liver from toxins, to reduce platelet aggregation, to prevent or treat inflammatory disease, inflammation, arthritis, psoriasis, cancer (e.g., prostate cancer and breast cancer), pain, Alzheimer's Disease, cardiovascular disease (e.g., arteriosclerosis and atherosclerosis). Turmeric extract that is standardized to 95% curcuminoid contains turmeric (with 95% curcuminoid).

In certain embodiments, curcuminoids prevent structural and behavioral changes of medial prefrontal cortex induced by sleep deprivation. Further, curcuminoids, as antioxidants decreases the production of acetaldehyde and free radicals from alcohol metabolism and increases activity of aldehyde dehydrogenase for increasing conversion of acetaldehyde to acetate.

The curcuminoid may not be soluble in water and therefore may exhibit poor bioavailability in humans and animals. As introduced above, the curcuminoid exhibits improved solubility in the solubilizing matrix which may result in improved bioavailability in humans and animals. Alternatively, or in addition to the solubilizing matrix, the flavonoid may include a water-soluble complex of curcuminoid for improving solubility in the pharmaceutically acceptable carrier which may result in improved bioavailability in humans and animals.

The composition may include the curcuminoid in an amount of from about 1 to about 10,000 mg, alternatively from about 10 to about 5,000 mg, or alternatively from about 100 to about 1,000 mg, based on a total weight of 100,000 mg for the composition. The curcuminoid is commercially available.

In other embodiments, a metabolite of curcumin, such as tetrahydrocurcumin and octahydrocurcumin, may be included in the composition. The composition may include the metabolite of curcumin in an amount of from about 1 to about 10,000 mg, alternatively from about 10 to about 5,000 mg, or alternatively from about 100 to about 1,000 mg, based on a total weight of 100,000 mg.

### Vitamin B Compounds

In various embodiments, the composition further includes a vitamin B compound. A complex of B vitamins including vitamins B1, B2, B6 and B12 may be used in the embodiments of the composition. In certain embodiments, the vitamin B compound includes vitamin B6, vitamin B12, or a combination thereof.

Elevated cortisol levels may be overcome by supplementing with vitamin B and vitamin C which may become depleted with prolonged hyperactivity of adrenal gland activity and increased production of cortisol.

Vitamin B-12 has been found to improve sleep-wake rhythm disorders. Likewise, vitamin B-6 deficiency has been linked to increased sleep disturbance and, thus, vitamin B-6 has been found to improve sleep by reducing such disturbance. Further, supplements containing B-1, B-6, and B-12 are cited to reduce hangover symptoms, as these B vitamins may be critical to metabolism of a subject. Since alcohol is a diuretic, causing one to use the restroom frequently, this frequent urination causes a loss of water and vitamins. These B vitamins are depleted as a result of consuming alcohol at least in part due to frequent urination.

The dose of each B vitamin used may, for example, be in the range of 1 to 100,000-times, 1 to 10,000-times, 1 to 1,000-times, 1 to 100-times, 1 to 50-times, 1 to 40-times, 1 to 30-times, or 1 to 20-times, the daily recommended dietary allowance (RDA) for the vitamin, such as at or around 10 or 20-times the RDA of each B vitamin used. The RDA for each vitamin, as defined herein, is that recommended by the National Academy of Sciences, Institute of Medicine, Food and Nutrition Board for 19-30 year old males, specifically: vitamin B-1 (thiamine) 1.2 mg/day; vitamin B-2 (riboflavin) 1.3 mg/day; vitamin B-6 (pyridoxine) 1.3 mg/day; and vitamin B-12 (cyanocobalamine) 2.4 micrograms/day. The dosages of each B vitamin used may, for example, be as follows: vitamin B-1 (thiamine) 100 mg; vitamin B-2 (riboflavin) 100 mg; vitamin B-6 (pyridoxine) 100 mg; and vitamin B-12 (cyanocobalamine) 100 micrograms.

### Ionic Liquid

The composition may include the ionic liquid. The ionic liquid may be utilized as the pharmaceutically acceptable carrier. Further, the ionic liquid may include the magnesium-containing compound. In various embodiments, the ionic liquid may be defined as compounds that consist quasi-exclusively of ions with a melting point below 100° C. as determined according to ASTM E794-06(2018). Various examples of ionic liquids may be found in Halayqa, Mohammed, et al. (2019). Polymer-Ionic liquid-Pharmaceutical conjugates as drug delivery systems. Journal of Molecular Structure. doi: 10.1016/j.molstruc.2018.12.023, Nuang, Weizi, et al. (2019). Ionic liquids: green and tailor-made solvents in drug delivery. Drug Discovery Today. doi: 10.1016/j.drudis.2019.09.018, Egorova, Ksenia, et al. (2017). Biological Activity of Ionic Liquids and Their Application in Pharmaceutics and Medicine. Chemical Reviews. doi: 10.1021/acs.chemrev.6b00562, and Marrucho, I. M., et al. Ionic Liquids in Pharmaceutical Applications. Annu. Rev. Chem. Biomol. Eng. doi: 10.1146/annurev-chembioeng-060713-040024, which are incorporated by reference as if fully set forth herein.

In certain embodiments, the ionic liquid includes ionic magnesium derived from a trace mineral concentrate. The ionic liquid may be present in the composition in any amount suitable as known in the art for facilitating delivery of the components/compounds of the composition described herein.

### Magnesium-Containing Compounds

The magnesium-containing compound may include a complex of magnesium and an anion selected from the group of chloride, taurinate, lactate, gluconate, citrate, malate, succinate, sulfate, propionate, hydroxide, oxide, orotate, phosphate, borate, salicylate, carbonate, bromide, stearate, an amino acid, butyrate, aspartate, ascorbate, picolinate, pantothenate, nicotinate, benzoate, phytate, caseinate, palmitate, pyruvate, threonate, and combinations thereof. Magnesium salts used with embodiments of the composition may, for example, provide a dose of elemental magnesium in the range of 50-500 mg, such as 100-300 mg, such as 200 mg. In various embodiments, the composition includes ionic magnesium derived from a trace mineral concentrate, magnesium citrate, magnesium aspartate, or combinations thereof, due to their high dissolution and consequently enhanced absorption of elemental magnesium. However, other magnesium salts may also be used. In certain embodiments, the magnesium-containing compound is derived from a trace mineral concentrate.

Magnesium plays a role in supporting deep, restorative sleep by maintaining healthy levels of GABA, a neurotransmitter that promotes sleep. Without being bound by theory, it is believed that magnesium can improve sleep quality, especially in people with poor sleep. Magnesium can also help insomnia that may be linked to the sleep disorder restless-leg syndrome. Magnesium also plays a key role in regulating the body's stress-response system by promoting GABA release. Further, magnesium may act as a stomach buffer and is an essential co-factor in a number of enzymatic processes that impact hangover from alcohol consumption. Magnesium is depleted as a result of consuming alcohol at least in part due to frequent urination.

The magnesium-containing compound may not be soluble in water and therefore may exhibit poor bioavailability in humans and animals. As introduced above, the magnesium-containing compound exhibits improved solubility in the solubilizing matrix which may result in improved bioavailability in humans and animals. Alternatively, or in addition to the solubilizing matrix, the composition may include a water-soluble complex of the magnesium-containing compound for improving solubility in the pharmaceutically acceptable carrier which may result in improved bioavailability in humans and animals.

Magnesium salts used with embodiments of the composition may, for example, provide a dose of elemental magnesium in the range of 50-500 mg, such as 100-300 mg, such as 200 mg.

### Coenzyme Q10

The composition may further in coenzyme Q10. Coenzyme Q10 is an essential electron and proton carrier that functions in the production of biochemical energy in aerobic organisms. Coenzyme Q10 is found in every cell in the body, thus its other name, ubiquinone (from the word ubiquitous and the coenzyme quinone). The structure of coenzyme Q10 consists of a quinone ring attached to an isoprene side chain. Because the body must have energy available to perform even the simplest operation, coenzyme Q10 is considered essential for the body's cells, tissues, and organs. Coenzyme Q10 also has antioxidant and membrane stabilizing properties that serve to prevent the cellular damage that results from normal metabolic processes. Even though the body has the ability to produce coenzyme Q10, deficiencies have been reported in a range of clinical conditions. Supplementation of the coenzyme helps guard against a possible deficiency. Aging is considered one reason for a deficiency, since the liver loses its ability to synthesize coenzyme Q10 as one gets older. Besides aging, poor eating habits, stress, and infection affect the body's ability to provide adequate amounts of coenzyme Q10. Known results of using coenzyme Q10 as an oral supplement are energy increase, improvement of heart function, prevention and cure of gum disease, a boost to the immune system, and possible life extension. The composition may include the coenzyme Q10 in any effective amount, such as from about 1 mg to about 10,000 mg, based on a total weight of 100,000 mg.

### Pharmaceutically Acceptable Carrier

The pharmaceutically acceptable carrier may be in the form of a solid, semi-solid, gelatinous, liquid, or combinations thereof. In certain embodiments, the composition is in an oral dosage form, such as a solid, semi-sold, gelatinous, or liquid oral dosage form. However, it is to be appreciated that the composition may be in any other dosage form known in the art.

When is a liquid dosage form, the pharmaceutically acceptable carrier may include water, oil, alcohol, or combinations thereof. The oral dosage form may be present in an amount of less than 100 milliliters. It is to be appreciated that powder/friable forms of the compositions and components may, for example, be mixed with food derived beverages, such as fruit juices, for oral administration by drinking the mixture.

When in a solid dosage form, the pharmaceutically acceptable carrier, for example, may be a compressed dosage form, such as a compressed tablet or caplet, obtained by compressing a powder mixture. The powder mixture may contain one or more active ingredients and optionally one or more excipients, such as binders, disintegrants, lubricants, fillers and the like, as known in the art.

Suitable fillers for solid compressed oral dosage forms include, but are not limited to, water-soluble compressible carbohydrates such as sugars, which include dextrose, sucrose, isomaltalose, fructose, maltose, and lactose, polydextrose, sugar-alcohols, which include mannitol, sorbitol, isomalt, maltitol, xylitol, erythritol, starch hydrolysates, which include dextrins, and maltodextrins, and the like, water insoluble plastically deforming materials such as microcrystalline cellulose or other cellulosic derivatives, water-insoluble brittle fracture materials such as dicalcium phosphate, tricalcium phosphate and the like and mixtures thereof. Suitable binders for solid compressed oral dosage forms include, but are not limited to, dry binders such as polyvinyl pyrrolidone, hydroxypropylmethylcellulose, and the like; wet binders such as water-soluble polymers, including hydrocolloids such as alginates, agar, guar gum, locust bean, carrageenan, tara, gum arabic, tragacanth, pectin, xanthan, gellan, maltodextrin, galactomannan, pusstulan, pullulan, laminarin, scleroglucan, gum arabic, inulin, pectin, whelan, rhamsan, zooglan, methylan, chitin, cyclodextrin, chitosan, polyvinyl pyrrolidone, cellulosics, starches, and the like; and derivatives and mixtures thereof.

Suitable disintegrants for solid compressed oral dosage forms include, but are not limited to, sodium starch glycolate, cross-linked polyvinylpyrrolidone, cross-linked carboxymethylcellulose, starches, microcrystalline cellulose, and the like.

Suitable lubricants for solid compressed oral dosage forms include, but are not limited to, long chain fatty acids and their salts, such as magnesium stearate and stearic acid, talc, and waxes.

Suitable glidants for solid compressed oral dosage forms include, but are not limited to, colloidal silicon dioxide, and the like.

### Emulsifier

The composition includes an emulsifier, which may form a solubilizing matrix or an emulsion to improve solubility and bioavailability of the compounds and components of the composition. In certain embodiments, an emulsion is a system made of at least two phases. Emulsions are generally homogeneous and stable. Examples of suitable emulsifiers include, but are not limited to, alginic acid, sodium alginate, potassium alginate, ammonium alginate, calcium alginate, propane-1,2-diol alginate, agar, carrageenan, processed eucheuma seaweed, locust bean gum, guar gum, tragacanth, acacia gum, xanthan gum, karaya gum, tara gum, konjac, pectins, cellulose derivatives selected from: methyl cellulose, hydroxypropyl cellulose, hydroxypropyl methyl cellulose, ethyl methyl cellulose, carboxy methyl cellulose, sodium carboxy methyl cellulose, crosslinked sodium carboxy methyl cellulose, enzymatically hydrolysed carboxy methyl cellulose, gelatine, and combinations thereof. In certain embodiments, the emulsifier includes glycerin, acacia gum, xanthan gum, or combinations thereof. The emulsifier may be included in the composition in any effective amount known in the art for solubilizing compounds. In various embodiments, the emulsifier dispersed throughout the pharmaceutically acceptable carrier to form a solubilizing matrix.

### Water-Soluble Complex

The composition may include water-soluble complexes of one or more of the components and compounds described herein. In various embodiments, some of the components and compounds of the composition may have low water solubility and may have low bioavailability. To increase water solubility and bioavailability, one or more of the compounds and components of the composition may be combined, individually or together, with a solubilizing compound to form the water-soluble complex. In certain embodiments, the solubilizing compound includes a cyclodextrin and the water-soluble complex of the compounds and components of the composition is formed from the cyclodextrin and the compounds and components. In certain embodiments, the cyclodextrin includes gamma cyclodextrin. Methods for utilizing the solubilizing compounds may be found in U.S Pat. App. Pub. No. 2010/0179103 B2, U.S. Pat. App. Pub. No. 2011/0034564 A1, and U.S. Pat. No. 9,731,015 B2, which are incorporated by reference as if fully set forth herein.

### Modified-Release Complex

The composition may include modified-release complexes of one or more of the components and compounds described herein. In various embodiments, it may be desirable for some of the components and compounds of the composition to be released to the body over a predetermined period of time or to be release to a specific target in the body. For example, it may be desirable for one or more of the components and compounds of the composition to exhibit a delayed release, a sustained release, or a controlled release. To modify release of one or more of the components and compounds of the composition, the one or more of the compounds and components of the composition may be combined, individually or together, with an encapsulating compound to form the modified-release complex. Examples of suitable encapsulating compounds include, but are not limited to, chitosans, starches, dextrins, cyclodextrins, celluloses, lignines, pectines, agar alginates, carragenatos, gelatins, guar gum, Arabic gum, tragacanth, lignosulfonates, Caravan gum, Ceratonia siliqua gum, saponines, Xanthan gums, seeds' gums, galactomanans, arabanogalactomanans, beta-glucanes, inulin, Psyllium, acacia gum, in all their isomeric and stereochemical configurations, in all their variations regarding quantity and quality of monomers or oligomers that constitute the hydrocolloid, in all their presentation forms, as metal, nitrogenated, phosphorated, sulfurated salts, as well all the derivatized products of the referred hydrocolloids. Other examples include compositions and methods for preparing a liquid, controlled-release formulation by blending one or more controlled release microbeads having one or more active agents, preparing a dense, thixotropic solution having a density that is at or about the density of the one or more microbeads, wherein a thixotropic agent, water and one or more preservatives are mixed under conditions that reduce bubble formation, e.g., using a mixer that lacks scraping paddles. The one or more microbeads may include an enteric coat, a resin coat, a lacquer coat, a pH-sensitive coating, a biodegradable polymer matrix, a water soluble matrix, an ionic matrix, combinations and mixtures thereof. The one or more microbeads may also includes one or more polymers selected from cellulose, ethylcellulose, methylcellulose, propylcellulose, methoxypropylcellulose, cellulose nitrate, poly(vinyl alcohol), poly(vinyl chloride), polystyrene, polyethylene, polypropylene, poly(ethylene-co-vinyl acetate), poly(hydroxybutyric acid), poly(hydroxyvalerianic acid-co-hydroxybutyric acid), poly(lactic acid), poly(glycolic acid), poly(lactic acid-co-glycolic acid), poly(ε(-caprolactones), poly(F-caprolactone-co-DL-lactic acid), poly(maleic anhydride), polyamides, gelatin, chitosan, collagen, poly(hydroxyalkyl)-L-glutamines, poly(γ-ethyl-L-glutaminate-co-glutamic acid), poly(L-leucine-co-L-aspartic acid), poly(proline-co-glutamic acid), poly(alkyl 2-cyanoacrylates), polyurethanes, poly(methyl methacrylate), poly(methyl methacrylate-co-methacrylic acid) and poly(methacrylate-co-hydroxypropyl methacrylate), polystyrene, polistirex, polacrilex and salts, combinations and mixtures thereof. Methods for utilizing the encapsulation compounds may be found in U.S. Pat. No. 8,911,783 B2 and U.S. Pat. App. Pub. No. 2013/0084340 A1, which are incorporated by reference as if fully set forth herein.

### Other Nutraceutical Compounds

The composition may include other nutraceutical compounds including, but not limited to, a mineral, black pepper extract, calcium, magnesium, chromium, copper, iodine, iron, manganese molybdenum, selenium, zinc, boron, sodium, potassium, silicon, an antioxidant, carotenoids, beta-carotene, lutein, zeaxanthin, lycopene, choline, para-aminobenzoic acid, alpha-lipoic acid, stilbenoid, resveratrol, an oil, an essential oil, a lipid, a salt, a phospholipid, linoleic acid, linolenic acid, inositol, methylsulfonyl methane, spirulina, a vitamin, vitamin A, vitamin C, vitamin D, vitamin E, vitamin K, pantothenic acid, pyridoxine, folic acid, biotin, derivatives thereof an amino acid, arginine, histidine, lysine, isoleucine, leucine, methionine, phenylalanine, threonine, tryptophan, tyrosine, valine, aspartate, glutamate, serine, proline, asparagine, glutamine, cysteine, glycine, alanine, ashwagandha, taurine. passion flower, lemon balm, American ginseng, ashwagandha (Withania somnifera), Asian ginseng, astragalus, cordyceps, reishi, eleutherococcus, holy basil, rhodiola, schisandra, maca, licorice, Magnolia officinalis, Phellodendron amurensewas, phosphatidylserine, L-theanine, and 4-amino-3-phenylbutyric acid.

In certain embodiments, the composition includes a cannabidiol (CBD), derivative, intermediate, or prodrug thereof. In an embodiment, the cannabidiol (CBD) may be a plant-extract, a synthetic compound, or a semi-synthetic compound.

Suitable cannabidiol (CBD) derivatives, intermediates, or prodrugs include, without limitation, Cannabigerol-type (CBG): cannabigerol ((E)-CBG C-5), cannabigerol monomethyl ether ((E)-CBGM C-5 A), Cannabinerolsaure A ((Z)-CBGA C-5 A), Cannabigerovarin (((e)-CBGV C-3), Cannabigerolsaure A (e)-CBGA C-5 A), A Cannabigerolsaure monomethylether ((e)-CBGAM C-5 A), Cannabigerovarinsaure A ((e)-CBGVA-C 3 A); Cannabichromene-type (CBC): cannabichromene (CBC-C 5), Cannabichromensaure A (CBCA C-5 A), Cannabichromevarin (CBCVC-3), Cannabichromevarinsaure A (CBCVAC3 A); Cannabidiol-type (CBD), cannabidiol (CBD-C 5), cannabidiol monomethyl (CBDM-C 5), cannabidiol-C4 (CBD-C 4), Cannabidivarin (CBDV-C 3), Cannabidiorcol (CBD-C 1), cannabidiolic (CBDA C-5), Cannabidivarinsaure (CBDVA C-3); Cannabinodiollike (CBND): Cannabinodiol (CBND C-5), Cannabinodivarin (CBND C-3); Cannabinol-type (CBN): Cannabinol CBNC 5, cannabinol C4 (CBN-C4), Cannabivarin (CBN-C 3), cannabinol C2 (CBNC 2), Cannabiorcol (CBN-C 1), Cannabinolsaure A (C 5 CBNA-A), Cannabinolmethylether (CBNM C-5) Cannabitriol-type (CBT): (−)-(9R, 10R)-trans-Cannabitriol ((−)-trans-CBTC 5), (+)-(9S, 10S)-Cannabitriol ((+)-trans-CBT C-5), (±)-(9R, 10S/9S, 10R)-Cannabitriol ((I)-cis-CBT-C 5), (−)-(9R, 10R)-trans [10-0-ethyl-cannabitriol] ((−)-trans-CBT-OEt-C 5), (±)-(9R, 10R/9S, 10S)-Cannabitriol-C3 ((±)-trans-CBT-C 3), 8,9-dihydroxy-A6a (10a) tetrahydrocannabinol (8,9-di-OH-CBT-C 5), cannabidiolic A (CBDA C-59-OH-CBT-C5 ester), (−)-(6aR, 9S, 10S, lOaR)-9, 10-dihydroxyhexahydrocannabinol, Cannabiripsol Cannabiripsol-C5, (−)-6a, 7, 10a-trihydroxy-A9-tetrahydrocannabinol ((−)-Cannabitetrol), 10-oxo-A6a (10a) tetrahydrocannabinol (OTHC); Cannabielsoin-like (CBE): (5aS, 6S, 9R, 9aR)-C 5-Cannabielsoin (CBEC-5), (5aS, 6S, 9R, 9aR)-C 3-Cannabielsoin (CBE C-3), (5aS, 6S, 9R, 9aR)-Cannabielsoinsaure A (CBEA-C 5 A), (5aS, 6S, 9R, 9aR)-Cannabielsoinsaure B (CBEA-C 5 B), (5aS, 6S, 9R, 9aR)-C3-Cannabielsoinsaure B (CBEA-C 3 B), Cannabiglendol-C3 (OH-iso-HHCV C-3), Dehydrocannabifuran (DCBF C-5), Cannabifuran (CBF-C 5); Isocannabinoide: (−)-Δ7-trans-(1R, 3R, 6R)-Isotetrahydrocannabinol, (±)-A7-1,2-cis-(1R, 3R, 6S/IS, 3S, 6R)-Isotetrahydrocannabivarin, (−)-A7-trans-(1R, 3R, 6R)-Isotetrahydrocannabivarin; Cannabicyclol-like (CBL): (±)-(laS, 3aR, 8bR, 8Cr-cannabicyclol (CBL-C 5), (±)-(laS, 3aR, 8bR, 8Cr-Cannabicyclolsaure A (CBLAC 5A) (±)-(laS, 3aR, 8bR, 8Cr-Cannabicyclovarin (CBLV C-3); Cannabicitran-type (CBT): Cannabicitran (CBT-C 5); Cannabichromanon-like (CBCN): Cannabichromanon (CBCN C-5), Cannabichromanon-C3 (CBCN C-3), Cannabicoumaronon (CBCON C-5), and combinations thereof. In a further embodiment, the cannabidiol (CBD), the derivative, the intermediate, or the prodrug thereof is selected from the group consisting of cannabidiolic acid (CBDA), cannabinol (CBN), cannabigerolic acid (CBGA), cannabinoid (CBG), cannabichromenic acid (CBCA), cannabichromene (CBC), cannabidivarin acid (CBDVA), cannabidivarin (CBDV), cannabigerovarin acid (CBGVA), and combinations thereof. In still a further embodiment, the cannabidiol (CBD), derivative, intermediate, or prodrug thereof is cannabidiol (CBD).

The composition may include a cannabidiol (CBD), derivative, intermediate, or prodrug thereof, and combinations thereof in an amount from about 0.5 mg/mL to about 25 mg/mL. In other embodiments, the composition comprises a cannabidiol (CBD), derivative, intermediate, or prodrug thereof, and combinations thereof in an amount of from about 1 mg/mL to about 25 mg/mL, from about 5 mg/mL to about 25 mg/mL, from about 5 mg/mL to about 20 mg/mL, from about 5 mg/mL to about 15 mg/mL, or from about from about 8 mg/mL to about 15 mg/mL. In some embodiments, the composition includes a cannabidiol (CBD), derivative, intermediate, or prodrug thereof, or combinations thereof in an amount of about 0.5 mg/mL, about 1 mg/mL, about 5 mg/mL, about 10 mg/mL, about 15 mL, about 20 mg/mL, or about 25 mg/mL.

In various embodiments, the compositions includes a tetrahydrocannabinol (THC), derivative, or intermediate thereof. In an embodiment, the tetrahydrocannabinol (THC) may be a plant-extract, a synthetic compound, or a semi-synthetic compound.

Suitable tetrahydrocannabinol (THC) derivatives or prodrugs include, without limit, Tetrahydrocannabinol-like (THC): A9-tetrahydrocannabinol (A9-THC-C 5), A9-tetrahydrocannabinol-C4 (A9-THC-C 4), A9-tetrahydrocannabivarin (A9-THCV-C 3), A9-Tetrahydrocannabiorcol (Δ9-THCO C-1), A9-Tetrahydrocannabinolsaure (Δ9 THCA-C-5 A), A9-Tetrahydrocannabinolsaure B (Δ9 THCA-C-5 B), A9-Tetrahydrocannabinolsaure-C4 (Δ9 THCA-C-4 A and/or B), Δ9-Tetrahydrocannabivarinsaure A (A9-THCVA-C 3 A), Δ9-Tetrahydrocannabiorcolsaure (A9-THCOA-C 1 A and/or B), (−)-A8-trans-(6aR, lOaR)-Δ8-tetrahydrocannabinol (A8-THC-C 5), (−)-A8-trans-(6aR, lOaR)-Tetrahydrocannabinolsaure A (A8-THCA-C 5 A); (−)-(6a S, 10a R)-Δ9-tetrahydrocannabinol ((−)-cis-A9-THC-C 5). In a further embodiment, the tetrahydrocannabinol (THC), derivative, or intermediate thereof is selected from the group consisting of tetrahydrocannabinolic acid (THCA), tetrahydrocannabivarin carboxylic acid (THCVA), tetrahydrocannabivarin (THCV), and combinations thereof. In still a further embodiment, the tetrahydrocannabinol (THC), derivative, or intermediate thereof is tetrahydrocannabinol (THC).

The composition may include a tetrahydrocannabinol (THC), derivative, or intermediate thereof, and combinations thereof in an amount of from about 0.5 mg/mL to about 30 mg/mL. In other embodiments, the composition comprises the tetrahydrocannabinol (THC), derivative, or intermediate thereof, and combinations thereof in an amount of from about 1 mg/mL to about 30 mg/mL, from about 2 mg/mL to about 30 mg/mL, from about 5 mg/mL to about 30 mg/mL, from about 5 mg/mL to about 25 mg/mL, from about 5 mg/mL to about 20 mg/mL, from about 5 mg/mL to about 15 mg/mL, or about 5 mg/mL to about 10 mg/mL. In some embodiments, the composition comprises the tetrahydrocannabinol (THC), derivative, or intermediate thereof, and combinations thereof in an amount of about 0.5 mg/mL, about 0.75 mg/mL, about 1 mg/mL, about 5 mg/mL, about 10 mg/mL, about 15 mL, about 20 mg/mL, about 25 mg/mL, or about 30 mg/mL.

In an embodiment, the composition may include a ratio of the cannabidiol (CBD), derivative, intermediate, or prodrug thereof, and combinations thereof to the tetrahydrocannabinol (THC), derivative, or intermediate thereof, and combinations thereof may be from about 25:1 to about 1:25. In some embodiments, the ratio of the cannabidiol (CBD), derivative, intermediate, or prodrug thereof, and combinations thereof to the tetrahydrocannabinol (THC), derivative, or intermediate thereof, and combinations thereof may be about 25:1, about 20:1, about 15:1, about 10:1, about 5:1, about 1:1, about 1:5, about 1:10, about 1:15, about 1:20, or about 1:25. In a further embodiment, the ratio of the cannabidiol (CBD), derivative, intermediate, or prodrug thereof, and combinations thereof to the tetrahydrocannabinol (THC), derivative, or intermediate thereof, and combinations thereof may be about 1:1. Other examples of nutraceutical compounds that may be included in the composition may be found in U.S. Pat. No. 8,734,855 B2 and PCT App. Pub. No. WO 2019/056128 A1, which are incorporated by reference as if fully set forth herein.

### Flavoring Agents

The composition may further include a flavoring agent. Examples of suitable flavoring agents include, but are not limited to, anethole, anise oil, benzaldehyde, blackberry, blueberry, caraway, caraway oil, cardamom oil, cardamom seed, cherry juice, cherry syrup, cinnamon, cinnamon oil, an alcohol, cinnamon water, citric acid, citric acid syrup, clove oil, cocoa, coriander oil, dextrose, eriodictyon, ethyl acetate, ethyl vanillin, fennel oil, ginger, glucose, glycerin, glycyrrhiza, grape, honey, lavender oil, lemon oil, lime, mannitol, methyl salicylate, myristica oil, orange oil orange peel, orange syrup, peppermint, peppermint oil, peppermint water, phenylethyl alcohol, pineapple, raspberry juice, raspberry syrup, rosemary oil, rose oil, rose water, sarsaparilla syrup, sorbitol, spearmint, spearmint oil, strawberry, sucrose, fructose, fruit juice, thyme oil, tolu balsam, vanilla, vanillin, wild cherry syrup, and combinations thereof.

### Other Conditions and Disorders

This composition can also be used for treating conditions or disorders, such as a disorder associated with C-reactive protein, autoimmune disease (e.g., multiple sclerosis, thyroiditis, rheumatoid arthritis, myositis, lupus, or Celiac disease), skin disease (e.g., eczema, urticaria, or psoriasis), lung disease (asthma, pulmonary fibrosis, or chronic obstructive pulmonary disease), prostatitis, arthritis, tumor, diabetes (type II diabetes), nonalcoholic fatty liver disease (NAFLD) via PPAR gamma antagonist activity, sexual dysfunction, chronic constipation, inflammatory disease (e.g., inflammatory bowel disease such as Crohn's disease or ulcerative colitis), infection, neurodegenerative disease (e.g., dyslexia, dyspraxia, autism, Asperger's disease, Alzheimer's disease, and mild cognitive impairment), and developmental disorder (e.g., attention deficit disorder or attention deficit hyperactivity disorder); for treating brain injury (e.g., physical damages to the brain); for improving concentration or mood; for improving the immune system, and for lowering blood cholesterol levels or blood pressure. “A disorder associated with C-reactive protein” refers to any disorder that results in an increase in the number of C-reactive protein in blood, such as inflammation or a cardiovascular disorder (e.g., atherosclerosis, coronary heart disease, stroke, and peripheral arterial disease). “Tumor” refers to both benign tumor and malignant tumor (e.g., leukemia, colon cancer, prostate cancer, kidney cancer, liver cancer, breast cancer, or lung cancer). “Infection” includes viral, bacterial, parasitic, and other microbial infection. Examples of viral infection treatable by this composition include influenza (e.g., Avian influenza or infection with influenza A virus subtype H5N1), severe acute respiratory syndrome (SARS), human immunodeficiency virus (HIV) infection, herpes simplex virus infection, respiratory syncytial virus (RSV) infection, rhinovirus (e.g., human rhinovirus) infection, and coronavirus infection. The mechanism for treating viral infection by this composition can include early stage inhibition of viral reproduction by reduction of viral RNA or DNA (e.g., by inhibition of transcription, reverse transcription, and translation). Bacterial infection include infection by either gram+ or gram− bacteria and infection by either anaerobic or aerobic bacteria. Examples of parasitic infection include leishmaniasis, malaria, and trypanosoma. Other examples of infection include respiratory infection, digestive tract infection, urinary tract infection, blood infection, and nervous system infection.

Further, this composition can be used to treat certain symptoms of the above-mentioned diseases or disorders. For example, it can be used to lessen certain symptoms of multiple sclerosis, including muscle weakness, wasting of muscles, pain (such as facial pain or pain without apparent cause), electrical shock sensation, loss of awareness of location of body parts, loss of coordination (such as in speech), shaking when performing fine movements, loss of ability to produce rapidly alternating movement (e.g., movement in a rhythm), and short-term or long term memory loss. As another example, it can be used to reduce the incidence, severity, and/or duration of cold and flu symptoms. In addition, this composition can also be used as a dietary supplement to improve the quality of life of a patient. For example, it can be used to reduce obesity (e.g., as a part a weight management plan), slow the aging process, enhance innate immunity, and improve skin health, sexual performance, and digestion.

In addition, this composition can be used to lessen negative side effects caused by chemotherapy with drugs such as, for example, glivec, taxol, and tamoxifen. Moreover, it is useful in enhancing athletic performance for both humans and animals, e.g., horses.

The terms “improving,” “enhancing,” “treating,” and “lowering” refer to the administration of an effective amount of a composition of the invention to a subject, who needs to improve one or more of the above-mentioned conditions or has one or more of the just-mentioned disorders, or a symptom or a predisposition of one of more of the disorders or conditions, with the purpose to improve one or more of these conditions, or to prevent, cure, alleviate, relieve, remedy, or ameliorate one or more of these disorders, or the symptoms or the predispositions of one or more of them. The term “administration” covers oral or parenteral delivery to a subject a composition of the invention in any suitable form, e.g., food product, beverage, tablet, capsule, suspension, gelatinous semi-solids, and solution. The term “parenteral” refers to subcutaneous, intracutaneous, intravenous, intramuscular, intraarticular, intraarterial, intrasynovial, intrasternal, intrathecal, intralesional, and intracranial injection, as well as various infusion techniques. An “effective amount” refers to a dose of the composition that is sufficient to provide a physical benefit (e.g., improving endurance) or a therapeutic benefit (e.g., lowering cholesterol or C-reactive protein levels, or reducing the risk of atherosclerosis or heart diseases). Both in vivo and in vitro studies can be conducted to determine optimal administration routes and doses.

The compositions described above can be preliminarily screened for their efficacy in treating the above-described conditions by in vitro assays and then confirmed by animal experiments and clinical trials. Other suitable analytical and biological assays are apparent to those of ordinary skill in the art. For example, the bioavailability of quercetin can be measured by conducting pharmacokinetic studies and evaluated by the area under the curve in a plasma-drug concentration time curve.

In general, whether the various compounds and components of the composition are synthetic or naturally derived, it should be understood that they are provided in and used in at least substantially pure forms with or without excipients and mixed or unmixed with one or more of the other active ingredients according to the invention as described herein. Thus, the compositions and components of the invention do not include foods and are not foods.

Also provided herein is a system and method for determining administration of the composition.

Although the invention has been described in connection with specific preferred embodiments, it should be understood that the invention as claimed should not be unduly limited to these specific embodiments. While at least one exemplary embodiment has been presented in the foregoing detailed description of the disclosure, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention. It being understood that various changes may be made in the function and arrangement of elements described in an exemplary embodiment without departing from the scope of the disclosure as set forth in the appended claims.

### Examples

### Prophetic Formulations

Table 1 below provides various prophetic examples of the composition.

| | Prophetic 1 | Prophetic 2 | Prophetic 3 | Prophetic 4 | Prophetic 5 |
|---|---|---|---|---|---|
| | (wt. %) | (wt. %) | (wt. %) | (wt. %) | (wt. %) |
| Flavonoid I | 2 | 2 | — | 2 | — |
| Water-soluble | — | — | 2 | — | 2 |
| Flavonoid I | | | | | |
| Mod-release | — | — | — | 2 | 2 |
| Flavonoid I | | | | | |
| Flavonoid II | 2 | 2 | — | 2 | — |
| Water-soluble | — | — | 2 | — | 2 |
| Flavonoid II | | | | | |
| Mod-release | — | — | — | 2 | 2 |
| Flavonoid II | | | | | |
| Mg-containing | 7 | 7 | — | 7 | — |
| compound | | | | | |
| Water-soluble | — | — | 7 | — | 7 |
| Mg compound | | | | | |
| Mod-release | — | — | — | 7 | 7 |
| Mg compound | | | | | |
| Prodrug of | 1 | 1 | 1 | 1 | 1 |
| cysteine | | | | | |
| Mod-release | — | — | — | 1 | 1 |
| Prodrug cys | | | | | |
| Amino acid | 1 | 1 | 1 | 1 | 1 |
| Mod-release | — | — | — | 1 | 1 |
| Amino acid | | | | | |
| Curcuminoid | 4 | — | — | 4 | — |
| Water-soluble | — | 4 | 4 | — | 4 |
| Curcuminoid | | | | | |
| Mod-release | — | — | — | 4 | 4 |
| Curcuminoid | | | | | |
| Vitamin B-6 | 0.1 | 0.1 | 0.1 | 0.1 | 0.1 |
| Mod-release | — | — | — | 0.1 | 0.1 |
| Vitamin B-6 | | | | | |
| Vitamin B-12 | 0.1 | 0.1 | 0.1 | 0.1 | 0.1 |
| Mod-release | — | — | — | 0.1 | 0.1 |
| Vitamin B-12 | | | | | |
| Emulsifiers | 4 | 4 | 4 | 4 | 4 |
| Additives | 1 | 1 | 1 | 2 | 2 |
| Carrier | balance | balance | balance | balance | balance |
| Total | 100 | 100 | 100 | 100 | 100 |

Table 2 below provides various additional prophetic examples of the composition including suitable, but non-limiting, ranges of the compounds and components.

| | Prophetic 6 | Prophetic 7 | Prophetic 8 | Prophetic 9 | Prophetic 10 |
|---|---|---|---|---|---|
| | (wt. %) | (wt. %) | (wt. %) | (wt. %) | (wt. %) |
| Flavonoid I | 0.1 to 20 | 0.1 to 20 | — | 0.1 to 20 | — |
| Water-soluble | — | — | 0.1 to 20 | — | 0.1 to 20 |
| Flavonoid I | | | | | |
| Mod-release | — | — | — | 0.1 to 20 | 0.1 to 20 |
| Flavonoid I | | | | | |
| Flavonoid II | 0.1 to 20 | 0.1 to 20 | — | 0.1 to 20 | — |
| Water-soluble | — | — | 0.1 to 20 | — | 0.1 to 20 |
| Flavonoid II | | | | | |
| Mod-release | — | — | — | 0.1 to 20 | 0.1 to 20 |
| Flavonoid II | | | | | |
| Mg-containing | 0.1 to 55 | 0.1 to 55 | — | 0.1 to 55 | — |
| compound | | | | | |
| Water-soluble | — | — | 0.1 to 55 | — | 0.1 to 55 |
| Mg compound | | | | | |
| Mod-release | — | — | — | 0.1 to 55 | 0.1 to 55 |
| Mg compound | | | | | |
| Prodrug of | 0.1 to 20 | 0.1 to 20 | 0.1 to 20 | 0.1 to 20 | 0.1 to 20 |
| cysteine | | | | | |
| Mod-release | — | — | — | 0.1 to 20 | 0.1 to 20 |
| Prodrug cys | | | | | |
| Amino acid | 0.1 to 20 | 0.1 to 20 | 0.1 to 20 | 0.1 to 20 | 0.1 to 20 |
| Mod-release | — | — | — | 0.1 to 20 | 0.1 to 20 |
| Amino acid | | | | | |
| Curcuminoid | 0.1 to 40 | — | — | 0.1 to 40 | — |
| Water-soluble | — | 0.1 to 40 | 0.1 to 40 | — | 0.1 to 40 |
| Curcuminoid | | | | | |
| Mod-release | — | — | — | 0.1 to 40 | 0.1 to 40 |
| Curcuminoid | | | | | |
| Vitamin B-6 | 0.01 to 1 | 0.01 to 1 | 0.01 to 1 | 0.01 to 1 | 0.01 to 1 |
| Mod-release | — | — | — | 0.01 to 1 | 0.01 to 1 |
| Vitamin B-6 | | | | | |
| Vitamin B-12 | 0.01 to 1 | 0.01 to 1 | 0.01 to 1 | 0.01 to 1 | 0.01 to 1 |
| Mod-release | — | — | — | 0.01 to 1 | 0.01 to 1 |
| Vitamin B-12 | | | | | |
| Emulsifiers | 0.1 to 40 | 0.1 to 40 | 0.1 to 40 | 0.1 to 40 | 0.1 to 40 |
| Additives | 0.1 to 10 | 0.1 to 10 | 0.1 to 10 | 0.1 to 10 | 0.1 to 10 |
| Carrier | balance | balance | balance | balance | balance |
| Total | 100 | 100 | 100 | 100 | 100 |

For both Table 1 and Table 2 above, the compounds and components are identified as follows:

Flavonoid I is a quercetin-containing compound, such as quercetin, which is commercially available.

Water-soluble Flavonoid I is a quercetin-containing compound complexed with a solubilizing compound, such as quercetin complexed with gamma cyclodextrin, which are individually commercially available.

Mod-release Flavonoid I is a quercetin-containing compound complexed with an encapsulating compound, such as quercetin complexed with an encapsulating compound, which are individually commercially available.

Flavonoid II is a GABA receptor antagonist, such as ampelopsin, which is commercially available.

Water-soluble Flavonoid II is a GABA receptor antagonist complexed with a solubilizing compound, such as ampelopsin complexed with gamma cyclodextrin, which is commercially available.

Mod-release Flavonoid II is a GABA receptor antagonist complexed with an encapsulating compound, such as ampelopsin complexed with an encapsulating compound, which are individually commercially available.

Mg-containing compound is a magnesium solution (100 mg/1 ml), derived from a trace mineral concentrate, which is commercially available.

Water-soluble Mg compound is a magnesium solution (100 mg/1 ml), derived from a trace mineral concentrate, the magnesium complexed with a solubilizing compound, such as the magnesium complexed with gamma cyclodextrin, which are commercially available.

Mod-release Mg compound is a magnesium solution (100 mg/1 ml), derived from a trace mineral concentrate, the magnesium complexed with an encapsulating compound, which are individually commercially available.

Prodrug of Cysteine is N-acetyl cysteine, which is commercially available.

Mod-release Prodrug Cys is N-acetyl cysteine complexed with an encapsulating compound, which are individually commercially available.

Amino acid is L-theanine, which is commercially available.

Mod-release amino acid is L-theanine complexed with an encapsulating compound, which are individually commercially available.

Curcuminoid is a curcuminoid including curcumin, which is commercially available.

Water-soluble Curcuminoid is a curcuminoid including curcumin, complexed with a solubilizing compound, such as a curcuminoid including curcumin complexed with gamma cyclodextrin, which are commercially available.

Mod-release Curcuminoid is a curcuminoid including curcumin, complexed with an encapsulating compound, which are individually commercially available.

Vitamin B-6 is a vitamin B-6 solution (8.5 mg/0.8 ml), which is commercially available.

Mod-release Vitamin B-6 is a vitamin B-6 solution (8.5 mg/0.8 ml), complexed with an encapsulating compound, which are individually commercially available.

Vitamin B-12 is a vitamin B-12 solution (5000 mcg/1 ml), which is commercially available.

Mod-release Vitamin B-12 is a vitamin B-12 solution (5000 mcg/1 ml), complexed with an encapsulating compound, which are individually commercially available.

Emulsifier includes a mixture of glycerin, acacia gum, and xanthan gum, which are individually commercially available.

Additives includes a mixture of flavoring agents, vitamin C, black pepper extract, honey, citric acid, and coenzyme Q10.

Carrier includes water.

### EXEMPLARY FORMULATIONS AND METHODS

### Example 1

Example 1: Actives Mixtures (Per 14 Doses)

1. Add 20 ml (100 mg/1 ml) of liquid magnesium to a 250 ml beaker to form an Actives Mixture
2. Add 70 ml of turmeric liquid (950 mg curcuminoid/15 ml) to the Actives Mixture
3. Add 22.4 grams (6 mg/0.8 ml) of Vitamin B1 to a 100 ml beaker to the Actives Mixtures, add 22.4 grams (6.5 mg/0.8 ml) of Vitamin B2 to the Actives Mixtures, add 22.4 grams (8.5 mg/0.8 ml) Vitamin B6 to the Actives Mixtures, and add 1 ml (5000 mcg/1 ml) of Vitamin B12 to the Actives Mixtures
4. Grind 7 pills of NAC (1000 mg/pill) into powder and combine with the Actives Mixture
5. Grind 14 pills of Quercetin/Bromelain (500 mg Quercetin/pill and 150 mg Bromelain/pill) into powder and combine with the Actives Mixture

Example 1: Gummy Mixture

1. Add 250 ml of orange juice to pot
2. Heat pot on stove at medium-low heat for 4 minutes
3. Place 500 ml beaker on hot plate
4. Add 1 gram of citric acid to 500 ml beaker, add 50 grams of honey to 500 ml beaker
5. Add stir bar to 500 ml beaker
6. Add thermocouple to 500 ml beaker
7. Transfer orange juice from pot to 500 ml beaker
8. Set temperature to 90° C.
9. Set rotation speed to 1000 RPM
10. Mix for 5 minutes
11. Add 75 grams of gelatin to 500 ml beaker to form a gelatin mixture
12. Add 7 ml of the gelatin mixture to each cell
13. Divide total weight of Actives Mixture by 14 to determine dose weight
14. Add the dose weight of the Actives Mixture to the center of each of the cells of the gelatin mixture using a pipette

### Example 2

Example 2: Actives Mixtures (Per 14 Doses)

1. Add 20 ml (100 mg/1 ml) of liquid magnesium to a 250 ml beaker
2. Add 70 ml of turmeric liquid to the 250 ml beaker
3. Add 1 ml (5000 mcg/1 ml) of Vitamin B12 to the 250 ml beaker
4. Add 600 mg of NAC to the 250 ml beaker
5. Add 500 mg of Quercetin Dihydrate to the 250 ml beaker

### 6. Add 400 mg of White Willow Bark Extract 15%

7. Grind 14 pills of Quercetin/Bromelain (500 mg Quercetin/pill and 150 mg Bromelain/pill) into powder and combine with the actives mixture

Example 2: Gummy Mixture (Same as Example 1)

### Example 3

Example 3: Actives Mixture (Per 50 Doses)

1. Add 87 g (66.5 mL) (100 mg/1 ml) of liquid magnesium to the beaker
2. Add 258 g (250 ml) (950 mg curcuminoid/15 ml) of turmeric liquid to the beaker
3. Add 1 ml (5000 mcg/1 ml) of Vitamin B12 to the beaker
4. Add 25000 mg of NAC to the beaker
5. Add 25000 mg of Quercetin Dihydrate to the beaker
6. Add 25000 mg of ampelopsin to the beaker
7. Set temperature to 45° C.
8. Set rotation speed to 1000 RPM
9. Mix for 5 minutes

Example 3: Liquid Carrier

1. Heat 750 ml of juice to 45° C.
2. Add 1 g of citric acid
3. Add 50 g of honey
4. Combine Liquid Carrier and Actives Mixture to form Composition
5. Mix Composition at 500 RPM for 1 hour at 45° C.
6. Divide into 50 vials

### Example 4

Example 4: Actives Mixture (Per 50 Doses)

1. Add 87 g (100 mg/1 ml) of liquid magnesium to a 500 ml beaker
2. Add 258 g (950 mg curcuminoid/15 ml) of turmeric liquid to the beaker
3. Set temperature to 55° C.
4. Set rotation speed to 700 rpm while waiting to reach 55° C.
5. Add 25 g of Quercetin Dihydrate to the beaker
6. Add 25 g of Ampelopsin to the beaker
7. Add 12.5 g of N-Acetyl Cysteine to the beaker

### 8. Add 12.5 g of L-Theanine

9. Add 1 ml Liquid B-12 (5000 mcg/1 ml)
10. Add 1 ml Liquid B-6 100 mg/1 ml)
11. Mix for 60 minutes at 55° C. at 950 rpm
12. Add 850 ml of Juice to a second beaker
13. Add 50 g of Honey to the second beaker
14. Heat second beaker to 55° C.
15. Add Juice/Honey mixture from the second beaker to the first breaker to form a Composition
16. Mix Composition for 50 minutes at 55° C. at 1000 rpm
17. Mix Composition for 10 minutes at 62° C. at 1000 rpm to pasteurize
18. Divide into 50 vials

### Experimental Testing

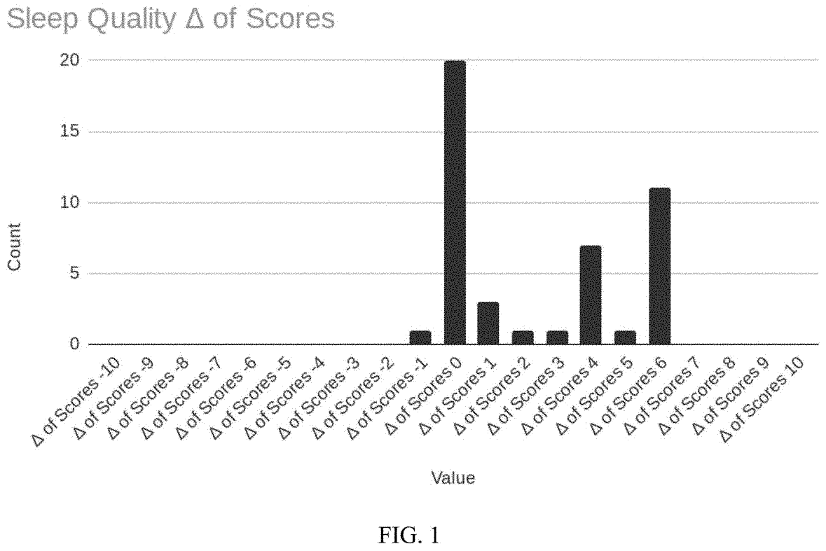

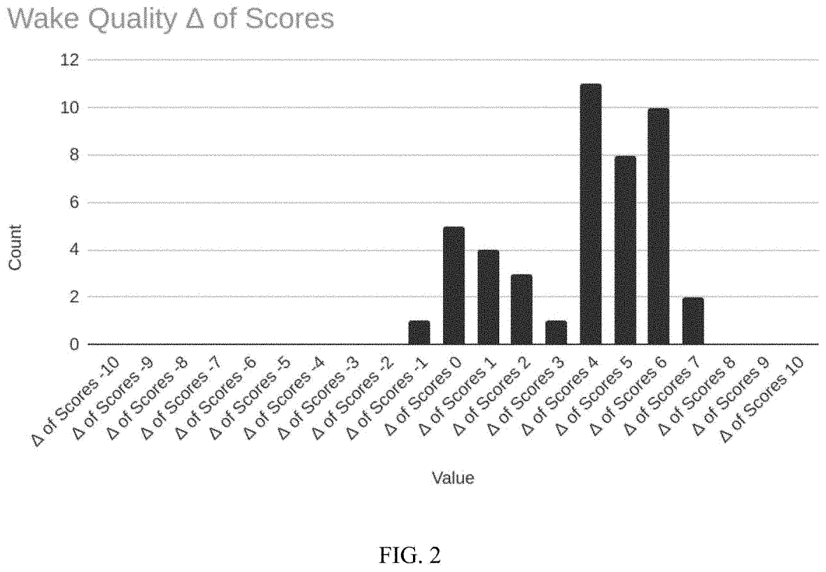

A composition according to Example 4 was prepared and administered to subjects having sleep-related indications. In particular, the composition was administered to the subjects 30 minutes before attempting to sleep. The subjects were asked to rate their sleep-quality without (comparative) and with (exemplary) the composition and wake-quality after sleeping without (comparative) and with (exemplary) the composition. Results of the analysis are provided below in Table 3. Results of the analysis are graphically shown in FIGS. 1 and 2.

TABLE 3

| | | Sleep Quality | Wake Quality | Sleep Quality | Wake Quality |
|---|---|---|---|---|---|
| | | without | without | with | with |
| Subject No. | Indication | Composition | Composition | Composition | Composition |
| 10001 | Night shift | 3 | 3 | 9 | 9 |
| 10001 | Night shift | 3 | 3 | 9 | 9 |
| 10001 | Night shift | 3 | 3 | 9 | 9 |
| 10001 | Night shift | 3 | 3 | 9 | 9 |
| 10001 | Night shift | 3 | 3 | 9 | 9 |
| 10001 | Night shift | 3 | 3 | 9 | 9 |
| 10001 | Night shift | 3 | 3 | 9 | 9 |
| 10001 | Night shift | 3 | 3 | 9 | 9 |
| 10001 | Night shift | 3 | 3 | 9 | 9 |
| 10001 | Night shift | 3 | 3 | 9 | 9 |
| 10003 | Night shift | 5 | 5 | 5 | 5 |
| 10004 | Night shift | 5 | 3 | 5 | 8 |
| 10004 | Night shift | 5 | 3 | 5 | 8 |
| 10004 | Night shift | 5 | 3 | 5 | 8 |
| 10004 | Night shift | 5 | 3 | 5 | 8 |
| 10005 | Night shift | 5 | 4 | 6 | 5 |
| 10006 | Night shift | 8 | 4 | 8 | 8 |
| 10006 | Night shift | 8 | 4 | 8 | 8 |
| 10006 | Night shift | 8 | 4 | 8 | 8 |
| 10006 | Night shift | 8 | 4 | 8 | 8 |
| 10006 | Night shift | 8 | 4 | 8 | 8 |
| 10008 | Insomnia | 3 | 5 | 9 | 8 |
| 10008 | Insomnia/Alcohol | 3 | 3 | 7 | 7 |
| 10008 | Insomnia | 3 | 5 | 6 | 7 |
| 10009 | Anxiety/Insomnia | 3 | 3 | 8 | 10 |
| 10009 | Anxiety/Insomnia | 4 | 6 | 8 | 10 |
| 10011 | Insomnia | 8 | 8 | 9 | 9 |
| 10012 | Night shift | 5 | 5 | 9 | 9 |
| 10014 | Restlessness/Headache | 8 | 7 | 9 | 9 |
| 10015 | Night shift | 6 | 2 | 8 | 9 |
| 10012 | Night shift | 5 | 5 | 9 | 9 |
| 10018 | Night shift | 5 | 6 | 5 | 7 |
| 10012 | Night shift | 5 | 5 | 9 | 9 |
| 10012 | Night shift | 5 | 5 | 9 | 9 |
| 10002 | Night shift | 6 | 6 | 5 | 5 |
| 10018 | Night shift | 5 | 6 | 5 | 7 |
| 10004 | Night shift | 5 | 3 | 5 | 8 |
| 10004 | Night shift | 5 | 3 | 5 | 8 |
| 10004 | Night shift | 5 | 3 | 5 | 8 |
| 10004 | Night shift | 5 | 3 | 5 | 8 |
| 10020 | Restlessness/Insomnia | 4 | 7 | 8 | 9 |
| 10019 | Night shift | 6 | 6 | 6 | 6 |
| 10019 | Night shift | 6 | 6 | 6 | 6 |
| 10021 | Night shift | 6 | 6 | 6 | 6 |
| 10021 | Night shift | 6 | 6 | 6 | 6 |

1. A composition for improving wellness of a human, the composition comprising:
a flavonoid;
a prodrug of cysteine; and
a pharmaceutically acceptable carrier comprising an ionic liquid having a melting point below 100° C. as determined according to ASTM E794-06(2018) to form a solubilizing matrix;
wherein the flavonoid exhibits an improved solubility in the pharmaceutically acceptable carrier in the presence of the solubilizing matrix as compared to solubility of a flavonoid in a pharmaceutically acceptable carrier substantially free of the solubilizing matrix.

2. The composition of claim 1, wherein the flavonoid comprises a quercetin-containing compound, a GABA(A) receptor antagonist, or a combination thereof.

3. The composition of claim 2, wherein the quercetin-containing compound is quercetin, a pharmaceutically acceptable salt thereof, a pharmaceutically active metabolite thereof, a pharmaceutically acceptable prodrug thereof, or a pharmaceutically acceptable solvate thereof.

4. The composition of claim 2, wherein the GABA(A) receptor antagonist is dihydromyricetin, a pharmaceutically acceptable salt thereof, a pharmaceutically active metabolite thereof, a pharmaceutically acceptable prodrug thereof, or a pharmaceutically acceptable solvate thereof.

5. The composition of claim 1, wherein the flavonoid comprises a water-soluble complex of a quercetin-containing compound, a water-soluble complex of a GABA(A) receptor antagonist, or a combination thereof.

6. The composition of claim 5, wherein the water-soluble complex is formed from an ionic liquid and the flavonoid.

7. (canceled)

8. The composition of claim 1 further comprising a curcuminoid.

9. (canceled)

10. The composition of claim 8, wherein the curcuminoid is curcumin, a pharmaceutically acceptable salt thereof, a pharmaceutically active metabolite thereof, a pharmaceutically acceptable prodrug thereof, or a pharmaceutically acceptable solvate thereof.

11. The composition of claim 1 further comprising a magnesium-containing compound, wherein the magnesium-containing compound comprises a complex of magnesium and an anion selected from the group consisting of chloride, taurinate, lactate, gluconate, citrate, malate, succinate, sulfate, propionate, hydroxide, oxide, orotate, phosphate, borate, salicylate, carbonate, bromide, stearate, an amino acid, butyrate, aspartate, ascorbate, picolinate, pantothenate, nicotinate, benzoate, phytate, caseinate, palmitate, pyruvate, threonate, and combinations thereof.

12. (canceled)

13. The composition of claim 11, wherein the magnesium-containing compound is ionic magnesium derived from a trace mineral concentrate.

14. The composition of claim 1, wherein the ionic liquid is ionic magnesium derived from a trace mineral concentrate.

15. The composition of claim 1, wherein the prodrug of cysteine comprises N-acetyl cysteine.

16. The composition of claim 1 further comprising a vitamin B compound, wherein the vitamin B compound comprises vitamin B-6, vitamin B-12, or a combination thereof.

17. (canceled)

18. The composition of claim 1, wherein the composition is in an oral dosage form that is liquid.

19. (canceled)

20. (canceled)

21. A composition for improving wellness of a human, the composition comprising:
a first flavonoid; and
a second flavonoid different from the first flavonoid;
wherein the composition is in the form of an oral dosage form that is liquid.

22. The composition of claim 21, wherein the first flavonoid comprises a quercetin-containing compound and the second flavonoid comprises a GABA(A) receptor antagonist.

23. A composition according to claim 21 for use in a method of treating, ameliorating, or preventing undesirable effects resulting from sleep deprivation.

24. A method for treating, ameliorating, or preventing undesirable effects resulting from sleep deprivation, the method comprising:
maintaining a wakeful state during a sleep-cycle period of a subject;
administering a composition according to claim 21 no greater than 2 hours before attempting to sleep; and
attempting to sleep.

25. A composition according to claim 21 for use in a method of treating, ameliorating, or preventing undesirable effects resulting from travel from one time zone to another time zone.

26. A method for treating, ameliorating, or preventing undesirable effects resulting from travel from one time zone to another time zone, the method comprising;
administering a composition according to claim 21 no greater than 2 hours before attempting to sleep; and
traveling from one time zone to another time zone.
